(19) United States
(12) Patent Application Publication
(10) Pub. No.: US 2024/0189227 A1
(43) Pub. Date: Jun. 13, 2024

(54) EASY CHEW FORMULATIONS

(71) Applicant: GlaxoSmithKline Consumer Healthcare Holdings (US) LLC, Wilmington, DE (US)

(72) Inventors: Shivangi Akash PATEL, Richmond, VA (US); Nikita Sandeep DESAI, Richmond, VA (US); Kenneth Earl ENGLAND, Richmond, VA (US); Than Van Nguyen PHAM, Richmond, VA (US)

(21) Appl. No.: 18/555,567

(22) PCT Filed: May 6, 2022
(86) PCT No.: PCT/US2022/027949

§ 371 Date: Oct. 16, 2023

Related U.S. Application Data
(60) Provisional application No. 63/185,469, filed on May 7, 2021.

(51) Int. Cl. A61K 9/00; A61K 31/485; A61K 47/02; A61K 47/10; A61K 47/12; A61K 47/26; A61K 47/36; A61K 47/44; A61K 47/46

(52) U.S. Cl. CPC A61K 9/0056; A61K 31/485; A61K 47/02; A61K 47/10; A61K 47/12; A61K 47/26; A61K 47/36; A61K 47/44; A61K 47/46

(57) ABSTRACT

A composition comprising an edible oil comprising coconut oil, palm kernel oil, palm oil, sunflower oil, hemp seed oil or any combination thereof; an edible triglyceride comprising cocoa butter, mango butter, ghee, hydrogenated palm oil, or any combination thereof; a humectant comprising glycerin, polyethylene glycol, propylene glycol, sorbitol, xylitol, maltitol, or any combination thereof; a binder comprising maltodextrin, sucrose, microcrystalline cellulose, pregelatinized starch, dextrose, lactose, or any combination thereof; and one or more flow aids.

### FIELD OF THE INVENTION

The invention is generally related to chewable tablets, and, more specifically, to soft or easy-chewable tablets with active pharmaceutical and nutritional ingredients.

### BACKGROUND

Soft chewable products are well-known and desirable pharmaceutical delivery forms. Soft chewable tablets are commonly used as an alternative to hard chewable tablets and capsule that must be swallowed, because the soft chewable tablets are generally tolerated by patients that are unable to consume hard chewable tablets or swallow capsules.

Compressed hard chewable tablets are one of the most common tablet forms. However, during chewing, these tablets fracture into a number of small particles to give a dry, gritty texture that is generally unfavored by consumers. An alternative design is an “easy chew” or “soft chew” formulation, which is generally has an amorphic, doughy, or semi-plastic consistency when chewed. These easy chew formulations advantageously lack the dryness and grittiness common in conventional hard chewable tablets.

Various “soft chew” formulations have been previously disclosed, but each suffer from one or more deficiencies. For example, often these formulations have unacceptable hardness levels of 3 kp or more, poor flow properties that make manufacturing difficult, long manufacturing processes, manufacturing processes that require high temperatures, or possess unpleasant tastes, texture, or other undesired physical characteristics.

### SUMMARY

In one aspect, a composition comprises an edible oil comprising coconut oil, palm kernel oil, palm oil, sunflower oil, hemp seed oil or any combination thereof; an edible triglyceride comprising cocoa butter, mango butter, ghee, hydrogenated palm oil, or any combination thereof; a humectant comprising glycerin, polyethylene glycol, propylene glycol, sorbitol, xylitol, maltitol, or any combination thereof; and a binder comprising maltodextrin, sucrose, microcrystalline cellulose, pregelatinized starch, dextrose, lactose, or any combination thereof.

In some embodiments, the composition comprises 0.01-16 wt. % edible oil. In some instances, the composition comprises 0.01-30 wt. % edible triglyceride. The composition comprises 2-15 wt. % humectant in some cases. In some embodiments, the composition comprises 20-70 wt. % of binder.

In some instances, the composition comprises 0.01-7 wt. % polyethylene glycol. The polyethylene glycol is PEG-400 to PEG-5000.

In some embodiments, the composition further comprises a flavorant.

The composition can further comprise one or more active pharmaceutical ingredients. In some cases, the active pharmaceutical ingredient comprises a pain or inflammation reducer, an antihistamine, a decongestant, a cough suppressant, a vitamin, a mineral, a biological active, a sleep aid, a stress-reducing aid, an energy and cognition aid, an Ayurveda supplement, an immunity supplement, a digestive aid, a joint supplement, a cannabinoid, a systemic natural, a nutritional agent, pharmaceutically acceptable salts thereof, or combinations thereof.

Exemplary pain or inflammation reducer comprises ibuprofen, naproxen, acetaminophen, salicylic acid, acetylsalicylic acid, ketoprofen, dexibuprofen, fenoprofen, dexketoprofen, flurbiprofen, oxaprozin, loxoprofen, diflunisal, etodolac, indomethacin, ketorolac, oxaprozin, piroxicam, salsalate, salicylic acid, indomethacin, tolmetin, sulindac, etodolac, ketordolac, diclofenac, aceclofenac, bromfenac, pharmaceutically acceptable salts thereof, or combinations thereof.

The antihistamine comprises cetirizine hydrochloride, levocetirizine hydrochloride, loratadine, desloratadine, fexofenadine hydrochloride, azelastine hydrochloride, olopatadine hydrochloride, brompheniramine maleate, chlorcyclizine hydrochloride, chlorpheniramine maleate, dexbrompheniramine maleate, dexchlorpheniramine maleate, diphenhydramine citrate, diphenhydramine hydrochloride, doxylamine succinate, phenindamine tartrate, pheniramine maleate, pyrilamine maleate, thonzylamine hydrochloride, triprolidine hydrochloride, pharmaceutically acceptable salts thereof, or combinations thereof.

The decongestant or cough suppressant comprises phenylephrine hydrochloride, pseudoephedrine hydrochloride, pseudoephedrine sulfate, phenylephrine bitartrate, dextromethorphan, pholcodine, codeine, benzonatate, pharmaceutically acceptable salts thereof, or combinations thereof.

The vitamin, mineral, or biological active comprises vitamin A, vitamin B, vitamin C, vitamin D, vitamin E, vitamin K, thiamin, riboflavin, niacin, folate, biotin, panthothenic acid, iron, phosphorus, iodine, copper, chromium, molybdenum, chloride, sodium, magnesium, calcium, zinc, selenium, manganese, potassium, choline, lycopene, lutein, L-threanine, coenzyme Q-10, phytosterols, hyaluronic acid, cognizing citicoline, silicon, icotinamide, Andrographis, pharmaceutically acceptable salts thereof, or combinations thereof.

The sleep aid comprises melatonin, lemon balm, lavender, chamomile, sage, valerian root extract, hops, passionflower extract, or combinations thereof.

The stress-reducing aid comprising sage, Ashwagandha (Withania somnifera), St. John's Wort, GABA (gamma-Aminobutyric acid), or combinations thereof.

The energy and cognition aid comprising green tea, ginseng, caffeine, rhodiola extract, B-vitamins, L-theanine, taurine, pharmaceutically acceptable salts thereof, or combinations thereof.

The Ayurveda supplement comprises amla (Emblica officinalis), bibhitaki (Terminalia bellirica), haritaki (Terminalia chebula)), Brahmi, cumin, licorice root, Gotu kolay, Cardamom, or combinations thereof.

The immunity supplement comprises copper, silver, spirulina, astragulus, beta glucan, aceola cherry extract, blood orange extract, elderberry, mushrooms (Lions Mane, Reishi, Shiitake, Cordyceps), pharmaceutically acceptable salts thereof, or any combinations thereof.

The digestive aid comprises a probiotic and combinations thereof; digestive enzymes; soluble fibers including psyllium husk, inulin, fenugreek fiber, wheat dextrin; Cascara sagrada; aloe ferox leaves extract, slippery elm bark extract, or combinations thereof.

The joint supplement comprises collagen, glucosamine, turmeric, methylsulfonylmethane (MSM), pharmaceutically acceptable salts thereof, or combinations thereof.

The cannabinoid comprises cannabidiol, cannabigerol, pharmaceutically acceptable salts thereof, and combinations thereof.

The systemic natural comprises one or more of Boswellia, curcumin, devils claw, ginger, feverfew, bromelain, turmeric, and butterbur.

The nutritional agent comprises black pepper, glycine, valerian root, eucalyptus, menthol, astragalus, bromelain, echinacea, white willow, ivy leaf (Hedera helix), ginger (Zingiber officinale), pharmaceutically acceptable salts thereof, or combinations thereof.

In some embodiments, the composition further comprises a solubility enhancer, a controlled release ingredient, a stabilizer, a taste masking ingredient, an antioxidant, a colorant, a plasticizer, or any combination thereof.

In some instances, the composition further comprises one or more flow aids. Exemplary flow aids include magnesium stearate, colloidal silicon dioxide, calcium stearate, stearic acid, glyceryl monostearate, glyceryl tribehenate, tricalcium phosphate, or any combination thereof. In some cases, the composition comprises up to 3 wt. % of one or more flow aids.

The composition can be a tablet in some embodiments. The tablet can have a hardness of less than 2 kp when tested with a conventional tablet hardness tester and a friability of less than 1.0% at 100 rotations.

In another aspect, a composition comprises 0.01-16 wt. % hydrogenated coconut oil; 0.01-14 wt. % cocoa butter; 2-15 wt. % glycerin; 0.01-7 wt. % polyethylene glycol; 20-70 wt. % maltodextrin, sucrose, and flavorant; and 0.1-3 wt. % flow aids. The polyethylene glycol is PEG 3350 in some instances. The flow aids comprise magnesium stearate and colloidal silicon dioxide in some cases.

In some embodiments, the composition comprises one or more active pharmaceutical ingredients previously recited herein.

The composition can be a tablet in some cases. The tablet can have a hardness of less than 2 kp when tested with a conventional tablet hardness tester and a friability of less than 1.0% at 100 rotations.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

All references to “percent weight,” “% wt.,” or “wt. %” are based on the total weight of the composition unless stated otherwise.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Described herein are compositions and methods of making such compositions that can be formed into easy/soft-chew tablets using traditional tablet presses. While various soft-chew formulations are known, these formulations suffer from poor processability (such as stickiness and low flowability), undesirable taste, and/or overly complicated methods of manufacture. Compositions and methods of making these compositions described herein

### I. COMPOSITIONS

In an aspect, a composition comprises one or more granulation aids; one or more intra-granulation aids; and one or more flow aids. In some embodiments, the granulation aid comprises an edible oil. In some cases, the edible oil has a high degree of saturation to prevent hardening and/or rancidity over time. The edible oil can have a naturally high level of saturation or can be a hydrogenated oil with the desired saturation level. Exemplary edible oils comprise coconut oil, palm kernel oil, palm oil, sunflower oil, hemp seed oil, or any combination thereof. Other edible oils not inconsistent with the objectives of this disclosure can also be used in some embodiments. Compositions described herein can comprise 0.01-16 wt. %, 0.01-15 wt. %, 0.01-14 wt. %, 0.01-13 wt. %, 0.01-12 wt. %, 0.01-11 wt. %, 0.01-10 wt. %, 0.01-9 wt. %, 0.01-8 wt. %, 0.01-7 wt. %, 0.01-6 wt. %, 0.01-5 wt. %, 0.01-4 wt. %, 1-16 wt. %, 2-16 wt. %, 3-16 wt. %, 4-16 wt. %, 5-16 wt. %, 6-16 wt. %, 7-16 wt. %, 8-16 wt. %, 9-16 wt. %, 10-16 wt. %, 11-16 wt. %, 12-16 wt. %, 13-16 wt. %, 14-16 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, or 16 wt. % edible oil.

The granulation aid can comprise or further comprise an edible triglyceride in some instances. Exemplary edible triglycerides include cocoa butter, mango butter, ghee, hydrogenated palm oil, or any combination thereof. In some embodiments, the edible oil is an edible triglyceride, or can comprise or further comprise an edible triglyceride. The composition can comprise 0.01-30 wt. %, 0.01-27 wt. %, 0.01-25 wt. %, 0.01-22 wt. %, 0.01-20 wt. %, 0.01-18 wt. %, 0.01-15 wt. %, 0.01-13 wt. %, 0.01-10 wt. %, 0.01-8 wt. %, 0.01-5 wt. %, 1-30 wt. %, 3-30 wt. %, 5-30 wt. %, 8-30 wt. %, 10-30 wt. %, 13-30 wt. %, 15-30 wt. %, 18-30 wt. %, 20-30 wt. %, 22-30 wt. %, 25-30 wt. %, 28-30 wt. %, 1-30 wt. %, 5-25 wt. %, 10-20 wt. %, 13-18 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, or 30 wt. % edible triglyceride in some embodiments.

The granulation aid can comprise a humectant in some embodiments. Typically, the humectant assists in moisture retention and drying prevention of the composition. In some instances, the humectant functions or additionally functions as a solvent. The humectant can comprise glycerin, polyethylene glycol (PEG), propylene glycol, sorbitol, xylitol, maltitol, or any combination thereof. The humectant can be present in the composition from 2-15 wt. %, 2-14 wt. %, 2-13 wt. %, 2-12 wt. %, 2-11 wt. %, 2-10 wt. %, 2-9 wt. %, 2-8 wt. %, 2-7 wt. %, 2-6 wt. %, 2-5 wt. %, 2-4 wt. %, 3-15 wt. %, 4-15 wt. %, 5-15 wt. %, 6-15 wt. %, 7-15 wt. %, 8-15 wt. %, 9-15 wt. %, 10-15 wt. %, 11-15 wt. %, 12-15 wt. %, 13-15 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. %.

In embodiments were the humectant comprises polyethylene glycol (PEG), the PEG is PEG-400-5000, 600-5000, 800-5000, 1000-5000, 1200-5000, 1400-5000, 1600-5000, 1800-5000, 2000-5000, 2500-5000, 3000-5000, 3500-5000, 4000-5000, 400-5000, 400-4500, 400-4000, 400-3500, 400-3000, 400-2500, 400-2000, 400-1500, 400-1000, 400-800, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000. When present, the composition can comprise 0.01-7 wt. %, 0.01-6 wt. %, 0.01-5 wt. %, 0.01-4 wt. %, 0.01-3 wt. %, 0.01-2 wt. %, 0.01-1 wt. %, 1-7 wt. %, 2-7 wt. %, 3-7 wt. %, 4-7 wt. %, 5-7 wt. %, 6-7 wt. %, 1 wt. %, 2 wt. %, 3 wt. %, 4 wt. %, 5 wt. %, 6 wt. %, or 7 wt. % PEG.

The intra-granulation aid can comprise one or more binders in some embodiments. In some cases, the binder comprises a polysaccharide. In some instances, the binder comprises maltodextrin, sucrose, microcrystalline cellulose, pregelatinized starch, dextrose, lactose, mannitol, calcium carbonate, or any combination thereof. The intra-granulation aid can be present in the composition in an amount of 20-70 wt. %, 20-65 wt. %, 20-60 wt. %, 20-55 wt. %, 20-50 wt. %, 20-45 wt. %, 20-40 wt. %, 20-35 wt. %, 20-30 wt. %, 20-25 wt. %, 25-70 wt. %, 30-70 wt. %, 35-70 wt. %, 40-70 wt. %, 45-70 wt. %, 50-70 wt. %, 55-70 wt. %, 60-70 wt. %, 65-70 wt. %, 25 wt. %, 30 wt. %, 35 wt. %, 40 wt. %, 45 wt. %, 50 wt. %, 55 wt. %, 60 wt. %, 65 wt. %, 70 wt. %.

Compositions herein can further comprise a flavorant. The flavorant can be any flavorant not inconsistent with the objectives of this disclosure, including, for example, a natural flavor, an artificial flavor, a bitterness masking agent, a natural sweetener, an artificial sweetener, an organic acid, a salt, honey, a granulated honey, a granulated syrup solid, a botanical extract, a botanical, a spice, cocoa, a chocolate, a sensate, or any combination thereof. In some instances, the flavorant can additionally function as a granulation aid, such as a chocolate which contains granulation aids such as cocoa butter and the like. The flavorant can be present between 0.01-15 wt. % in the composition.

Compositions described herein can further comprise one or more active pharmaceutical ingredients in some embodiments. The active pharmaceutical ingredient comprises a pain or inflammation reducer, an antihistamine, a decongestant, a cough suppressant, a vitamin, a mineral, a biological active, a sleep aid, a stress-reducing aid, an energy and cognition aid, an Ayurveda supplement, an immunity supplement, a digestive aid, a joint supplement, a cannabinoid, a systemic natural, a nutritional agent, pharmaceutically acceptable salts thereof, or combinations thereof.

The pain or inflammation reducer can comprise any pain or inflammation reducer not inconsistent with the objectives of this disclosure. Exemplary pain or inflammation reducer comprise ibuprofen, naproxen, acetaminophen, salicylic acid, acetylsalicylic acid, ketoprofen, dexibuprofen, fenoprofen, dexketoprofen, flurbiprofen, oxaprozin, loxoprofen, diflunisal, etodolac, indomethacin, ketorolac, oxaprozin, piroxicam, salsalate, salicylic acid, indomethacin, tolmetin, sulindac, etodolac, ketordolac, diclofenac, aceclofenac, bromfenac, pharmaceutically acceptable salts thereof, or combinations thereof.

The antihistamine can comprise any pain or inflammation reducer not inconsistent with the objectives of this disclosure. Exemplary antihistamines comprise cetirizine hydrochloride, levocetirizine hydrochloride, loratadine, desloratadine, fexofenadine hydrochloride, azelastine hydrochloride, olopatadine hydrochloride, brompheniramine maleate, chlorcyclizine hydrochloride, chlorpheniramine maleate, dexbrompheniramine maleate, dexchlorpheniramine maleate, diphenhydramine citrate, diphenhydramine hydrochloride, doxylamine succinate, phenindamine tartrate, pheniramine maleate, pyrilamine maleate, thonzylamine hydrochloride, triprolidine hydrochloride, pharmaceutically acceptable salts thereof, or combinations thereof.

The decongestant or cough suppressant can comprise any decongestant or cough suppressant not inconsistent with the objectives of this disclosure. Exemplary decongestant or cough suppressant comprises phenylephrine hydrochloride, pseudoephedrine hydrochloride, pseudoephedrine sulfate, phenylephrine bitartrate, dextromethorphan, pholcodine, codeine, benzonatate, pharmaceutically acceptable salts thereof, or combinations thereof.

The vitamin, mineral, or biological active can comprise any such vitamin, mineral, or biological active not inconsistent with the objectives of this disclosure. Exemplary vitamin, mineral, or biological actives comprise vitamin A, vitamin B, vitamin C, vitamin D, vitamin E, vitamin K, thiamin, riboflavin, niacin, folate, biotin, panthothenic acid, iron, phosphorus, iodine, copper, chromium, molybdenum, chloride, sodium, magnesium, calcium, zinc, selenium, manganese, potassium, choline, lycopene, lutein, L-threanine, coenzyme Q-10, phytosterols, hyaluronic acid, cognizing citicoline, silicon, icotinamide, Andrographis, pharmaceutically acceptable salts thereof, or combinations thereof.

The sleep aid can comprise any sleep aid not inconsistent with the objectives of this disclosure. Exemplary sleep aids comprise melatonin, lemon balm, lavender, chamomile, sage, valerian root extract, hops, passionflower extract, or combinations thereof.

The stress-reducing aid can comprise any stress-reducing aid not inconsistent with the objectives of this disclosure. Exemplary stress-reducing aids comprise sage, Ashwagandha (Withania somnifera), St. John's Wort, GABA (gamma-Aminobutyric acid), or combinations thereof.

The energy and cognition aid can comprise any energy and cognition aid not inconsistent with the objectives of this disclosure. Exemplary energy and cognition aids comprise green tea, ginseng, caffeine, rhodiola extract, B-vitamins, L-theanine, taurine, pharmaceutically acceptable salts thereof, or combinations thereof.

The Ayurveda supplement can comprise any Ayurveda supplement not inconsistent with the objectives of this disclosure. Exemplary Ayurveda supplements comprise amla (Emblica officinalis), bibhitaki (Terminalia bellirica), haritaki (Terminalia chebula)), Brahmi, cumin, licorice root, Gotu kolay, Cardamom, or combinations thereof.

The immunity supplement can comprise any immunity supplement not inconsistent with the objectives of this disclosure. Exemplary immunity supplements comprise copper, silver, spirulina, astragulus, beta glucan, aceola cherry extract, blood orange extract, elderberry, mushrooms (Lions Mane, Reishi, Shiitake, Cordyceps), pharmaceutically acceptable salts thereof, or any combinations thereof.

The digestive aid can comprise any digestive aid not inconsistent with the objectives of this disclosure. Exemplary digestive aids comprise a probiotic and combinations thereof; digestive enzymes; soluble fibers including psyllium husk, inulin, fenugreek fiber, wheat dextrin; (′ascara sagrada; aloe ferox leaves extract, slippery elm bark extract, or combinations thereof.

The joint supplement can comprise any joint supplement not inconsistent with the objectives of this disclosure. Exemplary joint supplements comprise collagen, glucosamine, turmeric, methylsulfonylmethane (MSM), pharmaceutically acceptable salts thereof, or combinations thereof.

The cannabinoid can comprise any cannabinoid not inconsistent with the objectives of this disclosure. Exemplary cannabinoids comprise cannabidiol, cannabigerol, pharmaceutically acceptable salts thereof, and combinations thereof.

The systemic natural can comprise any systemic natural not inconsistent with the objectives of this disclosure. Exemplary systemic naturals comprise one or more of Boswellia, curcumin, devils claw, ginger, feverfew, bromelain, turmeric, and butterbur.

The nutritional agent can comprise any nutritional agent not inconsistent with the objectives of this disclosure. Exemplary nutritional agents comprise black pepper, glycine, valerian root, eucalyptus, menthol, astragalus, bromelain, echinacea, white willow, ivy leaf (Hedera helix), ginger (Zingiber officinale), pharmaceutically acceptable salts thereof, or combinations thereof.

Active pharmaceutical ingredients can be present in the composition in amounts that are therapeutically or biologically active in a human or animal user as understood by a person of ordinary skill in the art. It will also be understood by a person of ordinary skill in the art that the amount or quantity of an active pharmaceutical ingredient necessary to be therapeutically or biologically active will be highly dependent on the unique and specific physical and biological properties of individual active pharmaceutical ingredients. In some instances, the active pharmaceutical ingredients can be present in less than therapeutically or biologically active amounts, such as nutraceutical or homeopathic dosing amounts.

Compositions described herein can comprise a solubility enhancer, a controlled release ingredient, a stabilizer, a taste masking ingredient, an antioxidant, a colorant, a plasticizer, or any combination thereof. Solubility enhancers are substances that enhance the solubility of one or more components in the composition. For instance, in some cases solubility enhancers can increase the solubility of an active pharmaceutical ingredient in the composition. Exemplary solubility enhancers include cyclodextrin, propylene glycol, polyethylene glycol, sugar alcohols, polyvinylpyrrolidone, or any combination thereof.

Controlled release ingredients can include any controlled release technology for active pharmaceutical ingredients not inconsistent with the objectives of this disclosure. Exemplary controlled release technology includes molecular or ionic barriers via complexation with active pharmaceutical ingredients, or by physical barriers for active pharmaceutical ingredients via coating or granulation. Complexation can, for example, include the use of an ion exchange resin, such as polystyrex ion resins complexed with an active pharmaceutical ingredient like dextromethorphan, or cyclodextrin and the like. Physical barriers can include encapsulation of an active pharmaceutical ingredient with a polymer, lipid, gelatin, and the like. Other controlled release ingredients can include enteric coatings, fatty acids, shellac, waxes, plastics, plant fibers. Further controlled release ingredient examples include hypromellose, ethylcellulose, cellulose acetate phthalate, alginates, medium chain triglycerides, zein, or any combination thereof.

Stabilizers generally contribute to chemical stability of one or more components in the composition. For example, some active pharmaceutical ingredients are stable only in environments with certain pH ranges, so a stabilizer could be a pH buffering system. Other exemplary stabilizers include povidone, polyvinyl alcohol, polyethylene glycol, hypromellose, hydroxypropyl cellulose, carboxymethylcellulose sodium, docusate sodium, sodium lauryl sulfate, polyethylene oxide, a food protein, an amino acid, co-polymers, or any combination thereof.

Many active pharmaceutical ingredients have bitter tastes that make oral delivery unpleasant. In some embodiments, compositions described herein can comprise a taste masking ingredient that hides or disguises the unpleasant taste of the active pharmaceutical ingredient to make the active more palatable. The taste masking ingredient can be any taste masking ingredient not inconsistent with the objectives of this disclosure. In some cases, the taste masking ingredient and the controlled release ingredient can be the same, such as the use of complexation or physical barriers. In other instances, the taste masking ingredient is unique and different from the controlled release ingredient and can be present in the absence of the controlled release ingredient. Exemplary taste masking ingredients include cyclodextrins, gelatin encapsulation, polymer encapsulation, sucralose, aspartame, stevia, congruent flavors, adenosine monophosphate, a lipoprotein, a phospholipid, sodium chloride, an organic acid, a salt of gluconic acid, a glycyrrhizate, or any combination thereof.

Compositions described herein can comprise one or more antioxidants. In some cases, the antioxidant is present in combination with an active pharmaceutical ingredient that is susceptible to oxidation to prevent oxidation and degradation of the active. Exemplary antioxidants include ascorbic acid, sodium ascorbate, calcium ascorbate, fatty acid esters of ascorbic acid, tocopherols, alpha-tocopherol, gamma-tocopherol, delta-tocopherol, propyl gallate, octyl gallate, dodecyl gallate, erythorbic acid, sodium erythorbate, dodecyl gallate, tertiary-butyl hydroquinone (TBHQ), butylated hydroxyanisole (BHA), butylated hydroxytoluene (BHT), citric acid, 4-hexylresorcinol, or any combination thereof.

Colorants can be any colorant known to those of ordinary skill in the art that are not inconsistent with the objectives of this disclosure.

Plasticizers are a class of compounds that can increase the flexibility of the composition, and can be present in compositions described herein to improve the “easiness” of the easy chew composition, the mouth feel of the composition, overall viscosity of the composition, and can prevent hardening of the composition over time. Exemplary plasticizers include a gelatin, a gum, glycerin, polysorbate 85/70, sortibal-sorbitan, or any combinations thereof. An exemplary gelatin can comprise a low bloom gelatin. An exemplary gum can comprise gum acacia, agar gum, guar gum, or any combination thereof. In some embodiments when gelatin and/or gum is present, compositions described herein can comprise a total of 0.01-5 wt. % gelatin, gum, or both.

Flow aids in the composition can increase the processability and manufacturing properties of the composition by improving flowability of the composition. As described in more detail herein, the composition can be granulated in some cases prior to compression into a tablet. To compress the composition into a tablet, the granulated composition is added to a hopper of a conventional tablet press. The flow aids improve the flowability of the granulated composition in the hopper by reducing clumping and sticking together of the granulated composition. Suitable flow aids can comprise any flow aid not inconsistent with the objectives of this disclosure. Exemplary flow aids comprise magnesium stearate, colloidal silicon dioxide, calcium stearate, stearic acid, glyceryl monostearate, glyceryl tribehenate, tricalcium phosphate, or any combination thereof. Notably, the flow aids can also function as lubricants, such as magnesium stearate. The composition can comprise up to 3 wt. %, up to 2 wt. %, up to 1 wt. %, 0.01-3 wt. %, 0.01-2.5 wt. %, 0.01-2 wt. %, 0.01-1.5 wt. %, 0.01-1 wt. %, 0.01-0.5 wt. %, 0.5-3 wt. %, 1-3 wt. %, 1.5-3 wt. %, 2-3 wt. %, or 2.5-3 wt. % of one or more flow aids.

Compositions described herein can be any dosage form not inconsistent with the objectives of this disclosure. In a preferred embodiment, the composition is a tablet. To qualify as an “easy” chew tablet, tablets made from the disclosed composition have a hardness of less than 1 kp, 2 kp, or 3 kp when tested with a conventional tablet hardness tester. Additionally, the tablets have a friability of less than 1.0% at 100 rotations, 200 rotations, or 300 rotations as tested on a conventional tablet friability tester.

The tablets can be non-coated or coated. Any coating known to those of ordinary skill in the art that is not inconsistent with the objectives of this disclosure can be used. In some cases, when the tablet is coated, the coating can prevent drying, oxidation, and/or water uptake of the tablet core. Furthermore, in some instances the coating can add flavoring, a crunchy exterior for texture contrast, or other desirable physical and/or observable characteristics.

### Example 1

### Compositions

In an exemplary embodiment, a composition comprised 0.01-16 wt. % hydrogenated coconut oil; 0.01-14 wt. % cocoa butter; 2-15 wt. % glycerin; 0.01-7 wt. % polyethylene glycol; 20-70 wt. % maltodextrin, sucrose, and flavorant; and 0.1-3 wt. % flow aids. The polyethylene glycol was PEG 3350.

The composition was further prepared with dextromethorphan as an API, with no physical changes being observed. These compositions are described in more detail in the Examples below.

The composition was compressed into tablets using a conventional tablet press. Each tablet had a hardness of less than 2 kp when tested with a conventional tablet hardness tester and a friability of less than 1.0% at 100 rotations.

### II. METHODS

### Multi-Pot Method

In another aspect, a method of preparing a composition described in Section I herein comprises combining all dry intra-granulation aid ingredients to form a first blend, reserving a first portion of the first blend; adding granulation aid ingredients to the first blend to form a combined blend; passing the combined blend through a sieve to form granules; coating the reserved first portion of the first blend onto the surface of the granules; passing the coated granules through the sieve a second time; and adding one or more flow aids to the coated granules to form a granulated mass. In cases where the granulation aid ingredients are solids, these granulation aid ingredients can be pre-heated until melting, and the melted liquids can be added to the first blend to form a combined blend.

In some embodiments, the method can further comprise combining intra-granulation aid ingredients and one or more active pharmaceutical ingredients to form the first blend. The active pharmaceutical ingredients can be one or more of the actives described in Section I herein.

In some instances, a portion of maltodextrin in reserved as the first portion rather than the first blend, and the reserved portion of maltodextrin is coated onto the surface of the granules.

The sieve size used for the method can be between 1000-3000 microns, 1200-3000 microns, 1400-3000 microns, 1600-3000 microns, 1800-3000 microns, 2000-3000 microns, 2200-3000 microns, 2400-3000 microns, 2600-3000 microns, 2800-3000 microns, 1000-2800 microns, 1000-2600 microns, 1000-2400 microns, 1000-2200 microns, 1000-2000 microns, 1000-1800 microns, 1000-1600 microns, 1000-1400 microns, 1000-1200 microns, 1000 microns, 1200 microns, 1400 microns, 1600 microns, 1800 microns, 2000 microns, 2200 microns, 2400 microns, 2600 microns, 2800 microns, or 3000 microns.

After formation of the granulated mass, the method can further comprise compressing the granulated mass on a tablet press to form a tablet having the physical properties described in Section I herein. The tablet press can be any conventional tablet press.

### Single Pot Method

In another embodiment, a one-pot method of preparing a composition described in Section I comprises combining all dry ingredients to form a pre-blend; adding one or more humectants to the pre-blend; sequentially adding granulation aids to pre-blend to form a blended mass; passing the blended mass through a sieve to form a granulated mass; and coating granulated mass with one or more flow aids. In some embodiment, the granulated mass is dried prior to coating with flow aids. In cases where the humectants and other granulation aid ingredients are solids, these ingredients can be pre-heated until melted, and the melted liquids can be added to the pre-blend. For example, solid coconut oil, cocoa butter, and PEG/glycerin can be pre-heated to melt the solids prior to addition to the pre-bend.

When incorporating one or more active pharmaceutical ingredients are incorporated into the composition, the method can further comprise combining the dry ingredients and one or more active pharmaceutical agents to form the first blend.

The sieve size used for the method can be between 1000-3000 microns, 1200-3000 microns, 1400-3000 microns, 1600-3000 microns, 1800-3000 microns, 2000-3000 microns, 2200-3000 microns, 2400-3000 microns, 2600-3000 microns, 2800-3000 microns, 1000-2800 microns, 1000-2600 microns, 1000-2400 microns, 1000-2200 microns, 1000-2000 microns, 1000-1800 microns, 1000-1600 microns, 1000-1400 microns, 1000-1200 microns, 1000 microns, 1200 microns, 1400 microns, 1600 microns, 1800 microns, 2000 microns, 2200 microns, 2400 microns, 2600 microns, 2800 microns, or 3000 microns.

The coated granulated mass can be compressed with a conventional tablet press to form a tablet having the physical properties described in Section I herein.

### Example 2

### Multi-Pot Method

Tablets having the composition described in Example 1 of the Section I herein were prepared using a two-pot method, as shown in Table 1.

TABLE 1
Placebo Composition

| | Ingredients | % w/w |
|---|---|---|
| Granular Addition | Hydrogenated Coconut Oil | 2 |
| | cocoa butter | 12 |
| | Glycerin | 12 |
| | Polyethylene Glycol 3350 | 2 |
| Intragranular Addition | Maltodextrin | 40.4 |
| | Flavor | 0.5 |
| | Powder sugar | 19.6 |
| Extra granular Addition | Maltodextrin | 10 |
| Flow Aid | Magnesium Stearate | 1 |
| | Colloidal Silicon Dioxide | 0.5 |

The dry intra-granulation aid ingredients were combined to form a first blend. The granulation addition ingredients were combined and added to the first blend to form a combined blend. Specifically, the PEG 3350 was combined with the glycerin, and each of the PEG/glycerin, hydrogenated coconut oil and cocoa butter were melted at approximately 40° C. prior to being added to the first blend. The combined blend was passed through a 10 #sieve (2000 microns) to form granules, and then the surface of the granules was coated with the extra-granular addition. The coated granules were passed through the 10 #sieve; and the flow aids were then additionally coated on the coated granules to form a granulated mass.

Finally, the granulated mass was loaded into a hopper on a conventional tablet press and the composition was compressed to form tablets having a hardness of less than 2 kp when tested with a conventional tablet hardness tester, and a friability of less than 1.0% at 100 rotations.

### Example 3

### One-Pot Method—Placebo Tablets

Tablets having a composition according to Table 2 were prepared using a one-pot method.

TABLE 2
Placebo Composition

| Ingredients | % w/w |
|---|---|
| Hydrogenated Coconut Oil | 6 |
| cocoa butter | 22 |
| Glycerin | 4 |
| Polyethylene Glycol 3350 | 2 |
| Maltodextrin | 50 |
| Flavorant | 2.76 |
| Powdered sugar | 9.44 |
| Granular corn syrup | 2.3 |
| Magnesium Stearate | 1 |
| Colloidal Silicon Dioxide | 0.5 |

First, all dry ingredients were combined to form a pre-blend having maltodextrin, flavor, and powdered sugar. Glycerin and polyethylene glycol were combined and added to the pre-blend to form an intermediate blend, and coconut oil and cocoa butter were sequentially added to the intermediate blend to form a blended mass. Specifically, the glycerin/PEG mixture was heated to approximately 40° C. to melt the PEG prior to adding to the pre-blend. The coconut oil and cocoa butter were similarly heated to approximately 40° C. to melt the solids prior to being added to the intermediate blend. The blended mass was then passed through a 10 #sieve (2000 micron) to form a granulated mass. The granulated mass was then dried and coated with magnesium stearate and colloidal silicone dioxide. Optionally the coated granulated mass can be passed a second time through the 10 #sieve. Finally, the coated granulated mass was loaded into a hopper on a conventional tablet press and the composition was compressed to form tablets having a hardness of less than 2 kp when tested with a conventional tablet hardness tester and a friability of less than 1.0% at 100 rotations.

### Example 4

### One-Pot Method—Dextromethorphan Tablets

Tablets were prepared from compositions having the active pharmaceutical ingredient dextromethorphan hydrobromide (“Dex”). To prepare these active containing tablets, Dex was combined with the dry ingredients to form the pre-blend, as described in more detail below.

TABLE 3
API-Containing Composition

| API formula | Ingredient | % w/w |
|---|---|---|
| API | Dextromethorphan HBr | 0.1786 |
| Granulation Aid | Hydrogenated Coconut Oil | 2 |
| | cocoa butter | 12 |
| | Glycerin | 12 |
| | Polyethylene Glycol 3350 | 2 |
| Intra Granulation | Maltodextrin | 40.4 |
| Aid | Sucrose | 17.44 |
| | Flavorant | 2.76 |
| Extra Granulation | Maltodextrin | 9.725 |
| aid | | |
| Flow aid | Magnesium Stearate | 1 |
| | Colloidal Silicon Dioxide | 0.5 |

First, all dry ingredients (including Dex) were combined to form a pre-blend having maltodextrin, flavor, powdered sugar, and Dex. Glycerin and polyethylene glycol were added to the pre-blend to form an intermediate blend, and coconut oil and cocoa butter were sequentially added to the intermediate blend to form a blended mass. Specifically, the glycerin/PEG mixture was heated to approximately 40° C. to melt the PEG prior to adding to the pre-blend. The coconut oil and cocoa butter were similarly heated to approximately 40° C. to melt the solids prior to being added to the intermediate blend. The blended mass was passed through a 10 #sieve (2000 micron) to form a granulated mass, which was then coated with the extra-granulation aid (maltodextrin). The coated granulated mass was then dried and coated with the flow aids magnesium stearate and colloidal silicone dioxide. Optionally the coated granulated mass can be passed a second time through the 10 #sieve. Finally, the coated granulated mass was loaded into a hopper on a conventional tablet press and the composition was compressed to form tablets having a hardness of less than 2 kp when tested with a conventional tablet hardness tester and a friability of less than 1.0% at 100 rotations.

### III. EMBODIMENTS

The skilled artisan would understand that modifications and variations of the compositions and methods described herein can be made within the scope of the invention. The following exemplary embodiments illustrate some, but not all, variations of the invention.
Embodiment 1. A composition comprising one or more granulation aids; one or more intra-granulation aids; and one or more flow aids.
Embodiment 2. The composition of Embodiment 1, wherein the granulation aid comprises an edible oil.
Embodiment 3. The composition of Embodiment 2, wherein the edible oil is coconut oil, palm kernel oil, palm oil, sunflower oil, hemp seed oil or any combination thereof.
Embodiment 4. The composition of Embodiments 2 or 3 comprising 0.01-16 wt. % edible oil.
Embodiment 5. The composition of any of Embodiments 1-4, wherein the granulation aid comprises an edible triglyceride.
Embodiment 6. The composition of Embodiment 5, wherein the edible triglyceride is cocoa butter, mango butter, ghee, hydrogenated palm oil, or any combination thereof.
Embodiment 7. The composition of Embodiments 5 or 6 comprising 0.01-30 wt. % edible triglyceride.
Embodiment 8. The composition of any of Embodiments 1-7, wherein the granulation aid comprises a humectant.
Embodiment 9. The composition of Embodiment 8, wherein the humectant comprises glycerin, polyethylene glycol, propylene glycol, sorbitol, xylitol, maltitol, or any combination thereof.
Embodiment 10. The composition of Embodiments 8 or 9 comprising 2-15 wt. % humectant.
Embodiment 11. The composition of Embodiment 9 comprising 0.01-7 wt. % polyethylene glycol.
Embodiment 12. The composition of Embodiments 9 or 11, wherein the polyethylene glycol is PEG-400 to PEG-5000.
Embodiment 13. The composition of any of Embodiments 1-12, wherein the intra-granulation aid comprises one or more binders.
Embodiment 14. The composition of Embodiment 13, wherein the binder comprises a polysaccharide.
Embodiment 15. The composition of Embodiment 13, wherein the binder comprises maltodextrin, sucrose, microcrystalline cellulose, pregelatinized starch, dextrose, lactose, or any combination thereof.
Embodiment 16. The composition of any of Embodiments 13-15 further comprising a flavorant.
Embodiment 17. The composition of any of Embodiments 13-16 comprising 20-70 wt. % of the intra-granulation aid.
Embodiment 18. The composition of any of Embodiments 1-17 further comprising one or more active pharmaceutical ingredients.
Embodiment 19. The composition of Embodiment 18, wherein the active pharmaceutical ingredient comprises a pain or inflammation reducer, an antihistamine, a decongestant, a cough suppressant, a vitamin, a mineral, a biological active, a sleep aid, a stress-reducing aid, an energy and cognition aid, an Ayurveda supplement, an immunity supplement, a digestive aid, a joint supplement, a cannabinoid, a systemic natural, a nutritional agent, pharmaceutically acceptable salts thereof, or combinations thereof.
Embodiment 20. The composition of Embodiment 19, wherein the pain or inflammation reducer comprises ibuprofen, naproxen, acetaminophen, salicylic acid, acetylsalicylic acid, ketoprofen, dexibuprofen, fenoprofen, dexketoprofen, flurbiprofen, oxaprozin, loxoprofen, diflunisal, etodolac, indomethacin, ketorolac, oxaprozin, piroxicam, salsalate, salicylic acid, indomethacin, tolmetin, sulindac, etodolac, ketordolac, diclofenac, aceclofenac, bromfenac, pharmaceutically acceptable salts thereof, or combinations thereof.
Embodiment 21. The composition of Embodiment 19, wherein the antihistamine comprises cetirizine hydrochloride, levocetirizine hydrochloride, loratadine, desloratadine, fexofenadine hydrochloride, azelastine hydrochloride, olopatadine hydrochloride, brompheniramine maleate, chlorcyclizine hydrochloride, chlorpheniramine maleate, dexbrompheniramine maleate, dexchlorpheniramine maleate, diphenhydramine citrate, diphenhydramine hydrochloride, doxylamine succinate, phenindamine tartrate, pheniramine maleate, pyrilamine maleate, thonzylamine hydrochloride, triprolidine hydrochloride, pharmaceutically acceptable salts thereof, or combinations thereof.
Embodiment 22. The composition of Embodiment 19, wherein the decongestant or cough suppressant comprises phenylephrine hydrochloride, pseudoephedrine hydrochloride, pseudoephedrine sulfate, phenylephrine bitartrate, dextromethorphan, pholcodine, codeine, benzonatate, pharmaceutically acceptable salts thereof, or combinations thereof.
Embodiment 23. The composition of Embodiment 19, wherein the vitamin, mineral, or biological active comprises vitamin A, vitamin B, vitamin C, vitamin D, vitamin E, vitamin K, thiamin, riboflavin, niacin, folate, biotin, panthothenic acid, iron, phosphorus, iodine, copper, chromium, molybdenum, chloride, sodium, magnesium, calcium, zinc, selenium, manganese, potassium, choline, lycopene, lutein, L-threanine, coenzyme Q-10, phytosterols, hyaluronic acid, cognizing citicoline, silicon, icotinamide, Andrographis, pharmaceutically acceptable salts thereof, or combinations thereof.
Embodiment 24. The composition of Embodiment 19, wherein the sleep aid comprises melatonin, lemon balm, lavender, chamomile, sage, valerian root extract, hops, passionflower extract, or combinations thereof.
Embodiment 25. The composition of Embodiment 19, wherein the stress-reducing aid comprising sage, Ashwagandha (Withania somnifera), St. John's Wort, GABA (gamma-Aminobutyric acid), or combinations thereof.
Embodiment 26. The composition of Embodiment 19, wherein the energy and cognition aid comprising green tea, ginseng, caffeine, rhodiola extract, B-vitamins, L-theanine, taurine, pharmaceutically acceptable salts thereof, or combinations thereof.
Embodiment 27. The composition of Embodiment 19, wherein the Ayurveda supplement comprises amla (Emblica officinalis), bibhitaki (Terminalia bellirica), haritaki (Terminalia chebula)), Brahmi, cumin, licorice root, Gotu kolay, Cardamom, or combinations thereof.
Embodiment 28. The composition of Embodiment 19, wherein the immunity supplement comprises copper, silver, spirulina, astragulus, beta glucan, aceola cherry extract, blood orange extract, elderberry, mushrooms (Lions Mane, Reishi, Shiitake, Cordyceps), pharmaceutically acceptable salts thereof, or any combinations thereof.
Embodiment 29. The composition of Embodiment 19, wherein the digestive aid comprises a probiotic and combinations thereof; digestive enzymes; soluble fibers including psyllium husk, inulin, fenugreek fiber, wheat dextrin; Cascara sagrada; aloe ferox leaves extract, slippery elm bark extract, or combinations thereof.
Embodiment 30. The composition of Embodiment 19, wherein the joint supplement comprises collagen, glucosamine, turmeric, methylsulfonylmethane (MSM), pharmaceutically acceptable salts thereof, or combinations thereof.
Embodiment 31. The composition of Embodiment 19, wherein the cannabinoid comprises cannabidiol, cannabigerol, pharmaceutically acceptable salts thereof, and combinations thereof.
Embodiment 32. The composition of Embodiment 19, wherein the systemic natural comprises one or more of Boswellia, curcumin, devils claw, ginger, feverfew, bromelain, turmeric, and butterbur.
Embodiment 33. The composition of Embodiment 19, wherein the nutritional agent comprises black pepper, glycine, valerian root, eucalyptus, menthol, astragalus, bromelain, echinacea, white willow, ivy leaf (Hedera helix), ginger (Zingiber officinale), pharmaceutically acceptable salts thereof, or combinations thereof.
Embodiment 34. The composition of any of Embodiments 1-33, further comprising a solubility enhancer, a controlled release ingredient, a stabilizer, a taste masking ingredient, an antioxidant, a colorant, a plasticizer, or any combination thereof.
Embodiment 35. The composition of any of Embodiments 1-34 further comprising one or more flow aids.
Embodiment 36. The composition of Embodiment 35, wherein the flow aid comprises magnesium stearate, colloidal silicon dioxide, calcium stearate, stearic acid, glyceryl monostearate, glyceryl tribehenate, tricalcium phosphate, or any combination thereof.
Embodiment 37. The composition of either 35 or 36 comprising up to 3 wt. % of one or more flow aids.
Embodiment 38. The composition of any of the preceding Embodiments, wherein the composition is a tablet.
Embodiment 39. The composition of Embodiment 38, wherein the tablet has a hardness of less than 2 kp when tested with a conventional tablet hardness tester and a friability of less than 1.0% at 100 rotations.
Embodiment 40. A composition comprising:
   0.01-16 wt. % hydrogenated coconut oil;
   0.01-14 wt. % cocoa butter;
   2-15 wt. % glycerin;
   0.01-7 wt. % polyethylene glycol;
   20-70 wt. % maltodextrin, sucrose, and flavorant; and
   0.1-3 wt. % flow aids.
Embodiment 41. The composition of Embodiment 40, wherein polyethylene glycol is PEG 3350.
Embodiment 42. The composition of Embodiments 40 or 41, wherein the flow aids comprising magnesium stearate and colloidal silicon dioxide.
Embodiment 43. The composition of any of Embodiments 40-42 further comprising an active pharmaceutical ingredient recited in Embodiment 19.
Embodiment 44. The composition of any of Embodiments 40-43, wherein the composition is a tablet.
Embodiment 45. The composition of Embodiment 44, wherein the tablet has a hardness of less than 2 kp when tested with a conventional tablet hardness tester and a friability of less than 1.0% at 100 rotations.
Embodiment 46. A method of preparing the composition of any of Embodiments 1-45 comprising: combining intra-granulation aid ingredients to form a first blend, reserving a first portion of the first blend; adding granulation aid ingredients to the first blend to form a combined blend; passing the combined blend through a sieve to form granules; coating the reserved first portion of the first blend onto the surface of the granules; passing the coated granules through the sieve; and adding one or more flow aids to the coated granules to form a granulated mass.
Embodiment 47. The method of Embodiment 46 further comprising combining intra-granulation aid ingredients and one or more active pharmaceutical agents to form the first blend.
Embodiment 48. The method of either Embodiment 46 or 47, wherein a portion of maltodextrin in reserved as the first portion rather than the first blend, and the reserved portion of maltodextrin is coated onto the surface of the granules.
Embodiment 49. The method of any of Embodiments 46-48, wherein the sieve size is between 1000-3000 microns.
Embodiment 50. The method of any of Embodiments 46-49, further comprising compressing the granulated mass on a tablet press to form a tablet.
Embodiment 51. A method of preparing the composition of any of Embodiments 1-45 comprising: combining all dry ingredients to form a pre-blend; adding one or more humectants to the pre-blend; sequentially adding oil and butter granulation aids to pre-blend to form a blended mass; passing the blended mass through a sieve to form a granulated mass; and coating granulated mass with one or more flow aids.
Embodiment 52. The method of Embodiment 51, further comprising drying the granulated mass prior to coating with flow aids.
Embodiment 53. The method of Embodiment 51 or 52 further comprising combining the dry ingredients and one or more active pharmaceutical agents to form the first blend.
Embodiment 54. The method of any of Embodiments 51-53, wherein the sieve size is between 1000-3000 microns.
Embodiment 55. The method of any of Embodiments 51-54, further comprising compressing the granulated mass on a tablet press to form a tablet.
Embodiment 56. A method of preparing the composition of any of Embodiments 40-43 comprising: combining all dry ingredients to form a pre-blend; adding glycerin and polyethylene glycol to the pre-blend; sequentially adding coconut oil and cocoa butter to the pre-blend to form a blended mass; passing the blended mass through a sieve to form a granulated mass; and coating granulated mass with magnesium stearate and colloidal silicone dioxide.
Embodiment 57. The method of Embodiment 56, wherein the dry ingredients comprise one or more active pharmaceutical ingredients.
Embodiment 58. The method of Embodiment 56 or 57, further comprising compressing the coated granulated mass on a tablet press to form a tablet.
Embodiment 59. The method of Embodiment 58, wherein the tablet has a hardness of less than 2 kp when tested with a conventional tablet hardness tester and a friability of less than 1.0% at 100 rotations.

1. A composition comprising:
an edible oil comprising coconut oil, palm kernel oil, palm oil, sunflower oil, hemp seed oil or any combination thereof;
an edible triglyceride comprising cocoa butter, mango butter, ghee, hydrogenated palm oil, or any combination thereof;
a humectant comprising glycerin, polyethylene glycol, propylene glycol, sorbitol, xylitol, maltitol, or any combination thereof; and
a binder comprising maltodextrin, sucrose, microcrystalline cellulose, pregelatinized starch, dextrose, lactose, or any combination thereof.

2. The composition of claim 1 comprising 0.01-16 wt. % edible oil.

3. The composition of claim 1 comprising 0.01-30 wt. % edible triglyceride.

4. The composition of claim 1 comprising 2-15 wt. % humectant.

5. The composition of claim 4 comprising 0.01-7 wt. % polyethylene glycol.

6. The composition of claim 5, wherein the polyethylene glycol is PEG-400 to PEG-5000.

7. The composition of claim 1 comprising 20-70 wt. % of binder.

8. (canceled)

9. The composition of claim 1 further comprising one or more active pharmaceutical ingredients.

10. The composition of claim 9, wherein the active pharmaceutical ingredient comprises a pain or inflammation reducer, an antihistamine, a decongestant, a cough suppressant, a vitamin, a mineral, a biological active, a sleep aid, a stress-reducing aid, an energy and cognition aid, an Ayurveda supplement, an immunity supplement, a digestive aid, a joint supplement, a cannabinoid, a systemic natural, a nutritional agent, pharmaceutically acceptable salts thereof, or combinations thereof.

11. The composition of claim 10, wherein the pain or inflammation reducer comprises ibuprofen, naproxen, acetaminophen, salicylic acid, acetylsalicylic acid, ketoprofen, dexibuprofen, fenoprofen, dexketoprofen, flurbiprofen, oxaprozin, loxoprofen, diflunisal, etodolac, indomethacin, ketorolac, oxaprozin, piroxicam, salsalate, salicylic acid, indomethacin, tolmetin, sulindac, etodolac, ketordolac, diclofenac, aceclofenac, bromfenac, pharmaceutically acceptable salts thereof, or combinations thereof.

12. The composition of claim 10, wherein the antihistamine comprises cetirizine hydrochloride, levocetirizine hydrochloride, loratadine, desloratadine, fexofenadine hydrochloride, azelastine hydrochloride, olopatadine hydrochloride, brompheniramine maleate, chlorcyclizine hydrochloride, chlorpheniramine maleate, dexbrompheniramine maleate, dexchlorpheniramine maleate, diphenhydramine citrate, diphenhydramine hydrochloride, doxylamine succinate, phenindamine tartrate, pheniramine maleate, pyrilamine maleate, thonzylamine hydrochloride, triprolidine hydrochloride, pharmaceutically acceptable salts thereof, or combinations thereof.

13. The composition of claim 10, wherein the decongestant or cough suppressant comprises phenylephrine hydrochloride, pseudoephedrine hydrochloride, pseudoephedrine sulfate, phenylephrine bitartrate, dextromethorphan, pholcodine, codeine, benzonatate, pharmaceutically acceptable salts thereof, or combinations thereof.

14. The composition of claim 10, wherein the vitamin, mineral, or biological active comprises vitamin A, vitamin B, vitamin C, vitamin D, vitamin E, vitamin K, thiamin, riboflavin, niacin, folate, biotin, panthothenic acid, iron, phosphorus, iodine, copper, chromium, molybdenum, chloride, sodium, magnesium, calcium, zinc, selenium, manganese, potassium, choline, lycopene, lutein, L-threanine, coenzyme Q-10, phytosterols, hyaluronic acid, cognizing citicoline, silicon, icotinamide, Andrographis, pharmaceutically acceptable salts thereof, or combinations thereof.

15. The composition of claim 10, wherein the sleep aid comprises melatonin, lemon balm, lavender, chamomile, sage, valerian root extract, hops, passionflower extract, or combinations thereof.

16. The composition of claim 10, wherein the stress-reducing aid comprising sage, Ashwagandha (Withania somnifera), St. John's Wort, GABA (gamma-Aminobutyric acid), or combinations thereof.

17. The composition of claim 10, wherein the energy and cognition aid comprising green tea, ginseng, caffeine, rhodiola extract, B-vitamins, L-theanine, taurine, pharmaceutically acceptable salts thereof, or combinations thereof.

18. The composition of claim 10, wherein the Ayurveda supplement comprises amla (Emblica officinalis), bibhitaki (Terminalia bellirica), haritaki (Terminalia chebula)), Brahmi, cumin, licorice root, Gotu kolay, Cardamom, or combinations thereof.

19. The composition of claim 10, wherein the immunity supplement comprises copper, silver, spirulina, astragulus, beta glucan, aceola cherry extract, blood orange extract, elderberry, mushrooms (Lions Mane, Reishi, Shiitake, Cordyceps), pharmaceutically acceptable salts thereof, or any combinations thereof.

20. The composition of claim 10, wherein the digestive aid comprises a probiotic and combinations thereof; digestive enzymes; soluble fibers including psyllium husk, inulin, fenugreek fiber, wheat dextrin; Cascara sagrada; aloe ferox leaves extract, slippery elm bark extract, or combinations thereof.

21. The composition of claim 10, wherein the joint supplement comprises collagen, glucosamine, turmeric, methylsulfonylmethane (MSM), pharmaceutically acceptable salts thereof, or combinations thereof.

22. The composition of claim 10, wherein the cannabinoid comprises cannabidiol, cannabigerol, pharmaceutically acceptable salts thereof, and combinations thereof.

23. The composition of claim 10, wherein the systemic natural comprises one or more of Boswellia, curcumin, devils claw, ginger, feverfew, bromelain, turmeric, and butterbur.

24. The composition of claim 10, wherein the nutritional agent comprises black pepper, glycine, valerian root, eucalyptus, menthol, astragalus, bromelain, echinacea, white willow, ivy leaf (Hedera helix), ginger (Zingiber officinale), pharmaceutically acceptable salts thereof, or combinations thereof.

25. The composition of claim 1 further comprising a solubility enhancer, a controlled release ingredient, a stabilizer, a taste masking ingredient, an antioxidant, a colorant, a plasticizer, a flavorant, a flow aid, or any combination thereof.

26. (canceled)

27. The composition of claim 25, wherein the flow aid comprises magnesium stearate, colloidal silicon dioxide, calcium stearate, stearic acid, glyceryl monostearate, glyceryl tribehenate, tricalcium phosphate, or any combination thereof.

28. The composition of claim 27 comprising up to 3 wt. % of one or more flow aids.

29. The composition of claim 1, wherein the composition is a tablet.

30. The composition of claim 29, wherein the tablet has a hardness of less than 2 kp when tested with a conventional tablet hardness tester and a friability of less than 1.0% at 100 rotations.

31. A composition comprising a tablet comprising:
0.01-16 wt. % hydrogenated coconut oil;
0.01-14 wt. % cocoa butter,
2-15 wt. % glycerin;
0.01-7 wt. % polyethylene glycol;
20-70 wt. % maltodextrin and sucrose; and
0.1-3 wt. % flow aids,
wherein the tablet has a hardness of less than 2 kp and a friability of less than 1.0% at 100 rotations.

32-36. (canceled)
